(19) United States
(12) Patent Application Publication
(10) Pub. No.: US 2013/0190640 A1
(43) Pub. Date: Jul. 25, 2013

(54) METHODS AND APPARATUS FOR THE MEASUREMENT OF PULMONARY PARAMETERS

(71) Applicants: Ori Adam, Rechovot (IL); Avi Lazar, Ra'anana (IL); Robert Shiner, Tel Aviv (IL); Zachi Peles, Tel Aviv (IL); Yehoshua Sheinman, Ra'anana (IL)

(72) Inventors: Ori Adam, Rechovot (IL); Avi Lazar, Ra'anana (IL); Robert Shiner, Tel Aviv (IL); Zachi Peles, Tel Aviv (IL); Yehoshua Sheinman, Ra'anana (IL)

(73) Assignee: PULMONE ADVANCED MEDICAL DEVICES, LTD., Ra'anana (IL)

(21) Appl. No.: 13/808,868

(22) PCT Filed: Jul. 6, 2011
(86) PCT No.: PCT/IL2011/000534

§ 371 Date: Apr. 17, 2013

Related U.S. Application Data
(60) Provisional application No. 61/361,680, filed on Jul. 6, 2010.

(51) Int. Cl. A61B 5/091; A61B 5/00; A61B 5/097; A61B 5/087

(52) U.S. Cl. CPC A61B 5/091; A61B 5/087; A61B 5/7278; A61B 5/097
USPC 600/538

(57) ABSTRACT

An apparatus includes a mouthpiece to communicate an airflow between a user and an airflow chamber in fluid communication with the mouthpiece; an interrupter in fluid communication with the airflow chamber and adapted to initiate an occlusion event by adjusting between an open state and a closed state, such that fluid communication is at least partially prevented between the airflow chamber and ambient air during the occlusion event initiated by the interrupter; a container in fluid communication with the airflow chamber; a flow rate sensor in fluid communication with the airflow chamber and adapted to measure an airflow exchange between the airflow chamber and the container during the occlusion event; and a controller adapted to determine an instantaneous lung volume based on the measured airflow exchange between the airflow chamber and the container.

### CLAIM OF PRIORITY

The present application claims the benefit of priority to U.S. Provisional Patent Application No. 61/361,680, filed on Jul. 6, 2010. The contents of the above application, as well as Application Serial No. PCT/IL2010/000070, are incorporated by reference as if fully set forth herein.

### TECHNICAL BACKGROUND

The present disclosure relates to methods and a device for measuring pulmonary function parameters and, more particularly, to a method and a device for calculating pulmonary function parameters according to pulmonary volumetric indicators.

### BACKGROUND

Absolute lung volume is a key parameter in pulmonary physiology and diagnosis but is not easy to measure in the live individual. It is relatively straightforward to measure the volume of air which is exhaled from a subject's mouth but at the end of complete exhalation, a significant amount of air is always left in the lungs because the mechanical properties of the lungs and chest wall, including the ribs, do not allow the lungs to collapse completely. The gas left in the lungs at the end of a complete exhalation is termed the Residual Volume (RV) which may be significantly increased or decreased in disease. The total volume of gas in the lungs at the end of a maximal inspiration is termed the Total Lung Capacity (TLC) which includes the RV plus the maximum amount of gas which can be inhaled or exhaled and which is termed the Vital Capacity (VC). However, during normal breathing the subject does not empty the lungs down to RV nor inflate them to TLC. The amount of gas in the lungs at the end of a normal breath, as distinct from a complete exhalation, is termed the Functional Residual Capacity (FRC) or Thoracic Gas Volume (TGV), depending upon the manner in which it is measured. For simplicity when this volume is measured by inert gas dilution techniques it will be termed FRC and when measured by barometric techniques involving gas compression as in this application it will be termed TGV.

In order to determine the total volumes of gas in the lungs at TLC, TGV or RV, indirect methods may be used since it is impossible to completely exhale all the gas from the lungs. Acceptable techniques for measuring lung volumes in humans include, for example: (1) Whole Body Plethysmography, in which a subject makes respiratory efforts against an obstruction within a gas tight chamber and the changes in pressure on the lung side of the obstruction can be related to the changes in pressure in the chamber through Boyle's law to calculate TGV; (2) Multi-breath Helium Gas Dilution involving the dilution of a known concentration and volume of Helium by the gas in the lungs of the subjects; (3) Nitrogen wash-out, in which upon the expiration of a known gas volume with 100% oxygen, the time required to resume normal atmospheric nitrogen concentrations is used to estimate lung volume; (4) Computerized Tomography, in which 3d imaging of the lungs are used to estimate lung volume; and (5) Chest Radiography, in which Lung volume is estimated from Chest radiography images. The most commonly used techniques, however, are gas dilution and whole body plethysmography.

Gas dilution involves the dilution of a known concentration and volume of inert gas by the gas in the lungs of the subjects and is critically dependent on complete mixing of the marker gas and lung gas. In subjects with poor gas mixing due to disease, this technique is very inaccurate and generally underestimates the true FRC. Whole body plethysmography, is generally believed to accurately measure TGV even in sick subjects but requires complicated and expensive equipment and is difficult to perform. Several studies, however, have shown that the whole body plethysmography may overestimate lung volumes in severely obstructed patients. For example, see O'Donnell, C. R., A. A. Bankier, L. Stiebellehner, J. J. Reilly, R. Brown, and S. H. Loring, Comparison of plethysmographic and helium dilution lung volumes: which is best for COPD? Chest, 2010. 137(5): p. 1108-15.

Once FRC (gas dilution), or TGV (whole body plethysmography), is calculated, the measurement by spirometry of the extra volume of gas which can be exhaled from the end of a normal exhalation (Expiratory Reserve Volume, ERV) and the extra volume which can be inhaled from the end of a normal exhalation (Inspiratory Capacity, IC) allows the calculation of TLC and RV.

These three important indicators (TLC, RV and FRC or TGV) are mutually connected through the following formulas: RV=FRC−ERV and TLC=FRC+IC and, TLC=RV+ERV+IC=RV+VC.

If FRC is determined by gas dilution and TGV by a barometric method, then the difference between them (TGV minus FRC) is a measure, albeit approximate, of the volume of poorly ventilated or “trapped gas” in the lungs.

In healthy subjects TGV and FRC should be virtually identical as there is little or no trapped gas, hence, for all practical matters, the term TGV shall apply for FRC as well. In summary, determination of TLC, TGV and RV is central to the complete evaluation of lung function.

### SUMMARY

In one general embodiment, an apparatus includes a mouthpiece adapted to communicate an airflow between a user and an airflow chamber in fluid communication with the mouthpiece; an interrupter in fluid communication with the airflow chamber and adapted to initiate an occlusion event by adjusting between an open state of the interrupter and a closed state of the interrupter, such that fluid communication is at least partially prevented between the airflow chamber and ambient air during the occlusion event initiated by the interrupter; a container in fluid communication with the airflow chamber; a flow rate sensor in fluid communication with the airflow chamber and adapted to measure an airflow exchange between the airflow chamber and the container during the occlusion event; and a controller adapted to determine an instantaneous lung volume based on the measured airflow exchange between the airflow chamber and the container.

In a first aspect according to the general embodiment, the controller is adapted to determine an instantaneous lung volume based only on the measured airflow exchange between the airflow chamber, container and ambient air.

In a second aspect according to any of the preceding aspects, the apparatus further includes a pump in fluid communication with the container.

In a third aspect according to any of the preceding aspects, the pump is adapted to initiate an airflow between the container and the atmosphere.

In a fourth aspect according to any of the preceding aspects, the interrupter is a first interrupter, and the apparatus further includes a second interrupter disposed between the airflow chamber and the container.

In a fifth aspect according to any of the preceding aspects, the second interrupter is adapted to initiate an occlusion event by adjusting between an open state of the second interrupter and a closed state of the second interrupter, such that fluid communication is at least partially prevented between the airflow chamber and the container during the occlusion event initiated by the second interrupter.

In a sixth aspect according to any of the preceding aspects, the apparatus further includes a first pressure sensor adapted to measure a static pressure of the container.

In a seventh aspect according to any of the preceding aspects, the pump is adapted to initiate an airflow from the container to the atmosphere when the first interrupter is in the open state and the second interrupter is in the closed state, such that the container is at a negative pressure relative to atmospheric.

In an eighth aspect according to any of the preceding aspects, the flow rate sensor is adapted to measure a first airflow exchange between the mouthpiece and the airflow chamber when the first interrupter is in the open state and the second interrupter is in the closed state.

In a ninth aspect according to any of the preceding aspects, the flow rate sensor is adapted to measure a second airflow exchange between the mouthpiece and the container when the second interrupter is adjusted from the closed state to the open state.

In a tenth aspect according to any of the preceding aspects, the controller is adapted to determine a difference between the first and second airflow exchanges.

In an eleventh aspect according to any of the preceding aspects, the controller is adapted to determine a level of flow limitation of the user based on the determined difference between the first and second airflow exchanges at the negative pressure in the container.

In a twelfth aspect according to any of the preceding aspects, at least one of the first or second interrupters is a shutter.

In a thirteenth aspect according to any of the preceding aspects, the apparatus further includes a second pressure sensor disposed within the airflow chamber and adapted to measure a static pressure of the airflow chamber.

In a fourteenth aspect according to any of the preceding aspects, the controller is adapted to receive a plurality of pressure measurements from the first and second pressure sensors.

In a fifteenth aspect according to any of the preceding aspects, the controller is adapted to determine a density of the measured airflow exchange based on the plurality of pressure measurements.

In a sixteenth aspect according to any of the preceding aspects, the controller is adapted to adjust the determined instantaneous lung volume based on the determined density of the measured airflow exchange.

In a seventeenth aspect according to any of the preceding aspects, the first interrupter is adapted to adjust to a state near but not at the closed state to initiate the occlusion event.

In an eighteenth aspect according to any of the preceding aspects, the controller is further operable to determine a residual volume of air in the lungs of the user based on the determined instantaneous lung volume.

In a nineteenth aspect according to any of the preceding aspects, the controller is further operable to determine a total lung capacity of the user based on the determined residual volume of air in the lungs of the user.

In a twentieth aspect according to any of the preceding aspects, the controller is further operable to determine a thoracic gas volume of the user based on the determined residual volume of air in the lungs of the user.

In a twenty-first aspect according to any of the preceding aspects, at least one of the airflow chamber or the container is adapted to be kept at substantially isothermal conditions.

In another general embodiment, a method includes receiving airflow through a conduit; occluding the conduit for a predetermined time interval to substantially prevent airflow through the conduit; measuring an airflow exchange between the conduit and a chamber in fluid communication with the conduit; and determining an instantaneous lung volume based on the measured airflow exchange.

In a first aspect according to the general embodiment, determining an instantaneous lung volume based on the measured airflow exchange includes determining an instantaneous lung volume based only on the measured airflow exchange.

In a second aspect according to any of the preceding aspects, the method further includes initiating a first occlusion event for a first predetermined time interval to substantially prevent airflow through the conduit.

In a third aspect according to any of the preceding aspects, the method further includes initiating a second occlusion event for a second predetermined time interval.

In a fourth aspect according to any of the preceding aspects, the method further includes concluding the first occlusion event.

In a fifth aspect according to any of the preceding aspects, the method further includes taking a plurality of pressure measurements in the conduit during the first and second occlusion events.

In a sixth aspect according to any of the preceding aspects, the method further includes determining an instantaneous lung volume based on the measured pressures.

In a seventh aspect according to any of the preceding aspects, the method further includes initiating a third occlusion event between the conduit and the chamber such that fluid communication is at least partially prevented between the conduit and the chamber.

In an eighth aspect according to any of the preceding aspects, the method further includes initiating an airflow between the chamber and the atmosphere during the third occlusion event.

In a ninth aspect according to any of the preceding aspects, the method further includes taking a pressure measurement of the chamber during the third occlusion event.

In a tenth aspect according to any of the preceding aspects, the method further includes concluding the third occlusion event.

In an eleventh aspect according to any of the preceding aspects, the method further includes measuring an airflow rate through the chamber subsequent to

In a twelfth aspect according to any of the preceding aspects, the method further includes determining a difference between the measured airflow exchange between the conduit and a chamber and the measured airflow rate through the chamber subsequent to concluding the third occlusion event.

In a thirteenth aspect according to any of the preceding aspects, the method further includes determining a level of flow limitation of the user based on the determined difference at the pressure measurement of the chamber taken during the third occlusion event.

In a fourteenth aspect according to any of the preceding aspects, the method further includes determining a density of the measured airflow exchange based on the plurality of pressure measurements.

In a fifteenth aspect according to any of the preceding aspects, the method further includes adjusting the determined instantaneous lung volume based on the determined density of the measured airflow exchange.

In a sixteenth aspect according to any of the preceding aspects, the method further includes determining a residual volume of air in the lungs of the user based on the determined instantaneous lung volume.

In a seventeenth aspect according to any of the preceding aspects, the method further includes determining a total lung capacity of the user based on the determined residual volume of air in the lungs of the user.

In an eighteenth aspect according to any of the preceding aspects, the method further includes determining a thoracic gas volume of the user based on the determined residual volume of air in the lungs of the user.

In a nineteenth aspect according to any of the preceding aspects, the method further includes maintaining at least one of the conduit or the chamber at substantially isothermal conditions.

Various embodiments of a breathing device and method for estimating an alveolar pressure according to the present disclosure may include one or more of the following features. For example, the breathing device may record an airway pressure during an entire breathing test, and in particular, during an interruption in a patient's breathing. The breathing device may also identify several instants during the interruption, such as an interruption initiation instant, an interruption termination instant, an instant of full airway and alveolar pressure equilibration, an instant of maximal positive deviation from base pressure and an instant of maximal negative deviation from base pressure. The breathing device may also estimate airway pressure at intermediate instants based on the identified instants, such as, for example, at an instant of interruption. As another example, the breathing device may also provide an accurate estimation on the user's alveolar pressure that would have existed in the absence of an interruption by, for example, reducing and/or eliminating effects on the estimated airway pressure due to compression or mechanical waves caused by an interrupter (e.g., a shutter). In addition, the breathing device may allow for a complete occlusion of a user's airway within a sufficiently short interval, allowing for an accurate determination of the alveolar pressure at the instant just prior to the interruption. The breathing device may also provide for an accurate calculation of lung parameters, such as lung volume, even under a relatively long time to achieve full occlusion of the airway.

Various embodiments of a breathing device and method for estimating an alveolar pressure according to the present disclosure may also include one or more of the following features. For example, the breathing device may provide for measurements of instantaneous pulmonary volume. The breathing device may measure instantaneous pulmonary volume based only on a flow rate of a respiratory airflow of a user between the airways of the user and an external container during a respiratory modulation, such as airway interruption. The breathing device may, therefore, allow for determination of a volumetric flow during the respiratory modulation based only on the measured flow rate. The breathing device may also provide for output of the instantaneous pulmonary volume for correlating pulmonary characteristics, such as TLC, RV, TGV, TV, lung compliance, airway resistance, and/or any combination thereof. As another example, the breathing device may also modulate a resistance to airflow of the user while the user's airways are occluded, for example, as a controlled volume change of the device that is in fluid communication with the airways of the user. The breathing device may thus provide for a derivation of an index of respiratory system instantaneous volume and compliance based on airway pressure changes resulting from an alteration of the modulation, in correlation with the calculated lung volume at the instant of the alteration.

In some disclosed embodiments, one principal advantage of a breathing device and method for estimating respiratory system volume according to the present disclosure is that confinement of the subject within a plethysmograph is not required. Further, the subject is not required to breathe special gases or be exposed to ionizing radiation. A breathing device and method for estimating pulmonary characteristics such as, but not restricted to, alveolar pressure, respiratory system volume and compliance, and airway resistance according to the present disclosure is also rapid and requires only minimal subject cooperation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

FIG. 1 is a schematic illustration of a breathing device for performing controlled occlusion of airways, according to some embodiments of the present disclosure;

FIG. 2 is a schematic illustration of a breathing device for performing controlled occlusion of airways connected to a user pulmonary system, according to some embodiments of the present disclosure;

FIG. 3 illustrates a graph that schematically depicts a time dependence of airflow recorded by an example embodiment of a breathing device for performing controlled occlusion of airways;

FIG. 4 illustrates a sealed container, partitioned into two volumes and containing an ideal gas, and shown in two different states;

FIG. 5 illustrates a continuous recording of a pulmonary volume change pertaining to a user;

FIG. 6 illustrates an example graph of gauge pressure reading over time during a sequential occlusion event;

FIG. 7 illustrates an example interface showing multiple panels displaying pressure vs. time, and volume flow rate vs. time, during a real occlusion event;

FIG. 8 is an illustration of an example breathing device for performing controlled occlusion of airways, according to some embodiments of the present disclosure;

FIG. 9 is an illustration of an example breathing assembly of a breathing device for performing controlled occlusion of airways, according to some embodiments of the present disclosure; and

FIG. 10 illustrates a comparison of results of measured TLC in patients using the whole body plethysmography technique vs. results of measured TLC in the same patients using a controlled occlusion of airways technique with a breathing device according to some embodiments of the present disclosure.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of the present disclosure. It will be understood by those of ordinary skill in the art that the present disclosure may be practiced without these specific details. In other instances, well-known methods, procedures, components and structures may not have been described in detail so as not to obscure the present disclosure.

The present disclosure is directed to a system and methods for determination of lung parameters, and more particularly, determination of Functional Residual Capacity (FRC) Thoracic Gas Volume (TGV), Total Lung Capacity (TLC) and Residual Volume (RV). The system and methods of the present application are designed to directly measure volume in the lungs with a desktop, portable station or handheld device, e.g., without the use of belts or large external chambers (e.g., exceeding 30 L). The principles and operation of a system and methods according to the present disclosure may be better understood with reference to the drawings and accompanying descriptions.

For example, according to some embodiments of the present disclosure, there is provided a device and method for estimating an alveolar pressure by interrupting a respiratory airflow of user breathing through a breathing apparatus. The method is based on a recording of an airway pressure in a breathing apparatus during an entire test, and in particular, during an interruption. The recording allows identifying a plurality of instants during the interruption, such as an interruption initiation instant, an interruption termination instant, an instant of full airway and alveolar pressure equilibration, an instant of maximal positive deviation from base pressure and an instant of maximal negative deviation from base pressure. The airway pressure during these instants allows estimating, optionally by interpolation or extrapolation, the airway pressure during other intermediate instants that occur during the interruption, such as estimated alveolar pressure at the instant of interruption, and the airway pressure rate of change with respect to time, such as the airway pressure rate of change with respect to time shortly prior or after the interruption.

The estimated airway pressure at the instant of occlusion may be negligibly affected by compression or mechanical waves that are caused by the interruption device and therefore provides an accurate estimation on the user's alveolar pressure that would have existed in the absence of an interruption. In addition, the complete occlusion of airways is optionally accomplished within a sufficiently short interval, allowing for an accurate determination of the alveolar pressure at the instant just prior to the interruption. Additionally, if airflow is allowed into a closed chamber following the interruption, necessary corrections to the recorded pressure due to the existence of flow may be added in order to estimate the pressure under static conditions. Such corrections are generally proportional to the square of the flow. Optionally, in some embodiments, an accurate calculation of lung parameters such as lung volume is achieved even when the time it takes to achieve full occlusion of the airways may is relatively long.

Additionally, according to some embodiments of the present disclosure, there is provided a method for measuring instantaneous pulmonary volume. Optionally, the density related pulmonary volume changes by this method may be used in correlation with the alveolar pressure changes in order to calculate the instantaneous pulmonary volume. The method may be based on a flow rate of a respiratory airflow of a user between the airways of the user and an external container during a respiratory modulation, such as airway interruption. The flow rate allows determining a volumetric flow during the respiratory modulation, for example by calculating the integral of the flow rate. The instantaneous pulmonary volume may then be outputted, either for presentation to the user or to a physician and/or for correlating pulmonary characteristics, such as TLC, RV, TGV, TV, lung compliance, airway resistance, and/or any combination thereof.

In some embodiments of the present disclosure, there is provided a device intended to alter the modulation, while airways are occluded. Optionally, the modulation alteration is in the form of a controlled volume change of a device that is in fluid communication with the airways of the subject. Further, an alteration of the modulation may be performed while airways are occluded. The airway pressure changes resulting from the alteration, in correlation with the calculated lung volume at the instant of the alteration are then used to derive an index of respiratory system instantaneous volume and compliance. According to some aspects, there is provided a number of processes for correlating pulmonary alveolar and/or density related pulmonary volume changes to pulmonary characteristics, such as TLC, RV, TGV, TV, lung compliance, airway resistance, and/or any combination thereof.

In some embodiments of the breathing device of the present disclosure, during a spontaneous inspiratory effort, an interrupter (e.g., shutter) is abruptly shut, and upon closure, gas flow (between the device and the atmosphere) is completely interrupted. Owing to ongoing effort of respiratory muscle efforts as well as inertia of respiratory tissues, some finite time may be required for chest wall and lung tissue to stop and system pressures to equilibrate. Indeed, for a very short time well before that equilibration can occur, tissue compartment flows that are attributable to ongoing tissue displacements can be considered as a high impedance flow source, and thereby act to rarify gas both in the thorax and in the external container, with mass conservation requiring that tissue compartment flows equal the sum of thoracic gas flows and flows of gas in an external chamber of the device is implied.

For a very short time period after shutter closure, the tissue compartment flow that is associated with ongoing tissue displacements is assumed to be well-approximated by the tissue compartment flow at the instant immediately before shutter closure. This tissue flow immediately before shutter closure is reported by a flow sensor, since flows attributable to gas rarefaction in either the thorax or the external chamber before shutter closure are altogether negligible. For sufficiently short time periods following shutter closure, tissue compartment flow may be equal to a flow reported by the flow sensor at the instant before shutter closure.

With the shutter closed, tissue compartment flow can be accommodated only by rarefaction of gas in the thorax and the external chamber. Gas compression and rarefaction in the lung is ordinarily assumed to be isothermal, and that in the external chamber for short times may be approximately adiabatic. Departures of a measured flow signal from the flow sensor from an ideal flow signal may be attributable in part to the effects of airway inertance, compliance and resistance; gas volume of the breathing device airways is small compared with gas volume of lung or external chamber.

If the thermodynamic properties of the external chamber gas are known, such as but not restricted to volume, airway resistance, and temperature, the thoracic gas volume at the instant of shutter closure can be calculated. TLC is then determined by adding the instantaneous inspiratory reserve volume measured spirometrically to the thoracic gas volume, as described in the present disclosure.

As used herein a “user” or “subject” means a healthy user, a subject, or a patient to whom the one or more instantaneous pulmonary measurements relating to the lungs, medical condition and/or respiration, are related. The user may be a person or an animal operating a breathing device, for example as described below, a healthy user. As used herein, an airway means an active airway that allows actively passing respiratory airflow, for example the mouth and/or one or more nostrils. The airway occlusion may be external, for example by using the breathing device 100 described below, which is external to the body and/or internally, for example in the mouth's lumen.

As used herein, a correlation means associating between instantaneous pulmonary properties of a user which are measured at the same respiratory instant and/or stage, mapping and/or binning instantaneous pulmonary properties of a user which are measured at the same respiratory instant and/or stage, and/or scaling and/or normalizing one instantaneous pulmonary property of a user according to another instantaneous pulmonary property which is measured at the same respiratory instant and/or stage.

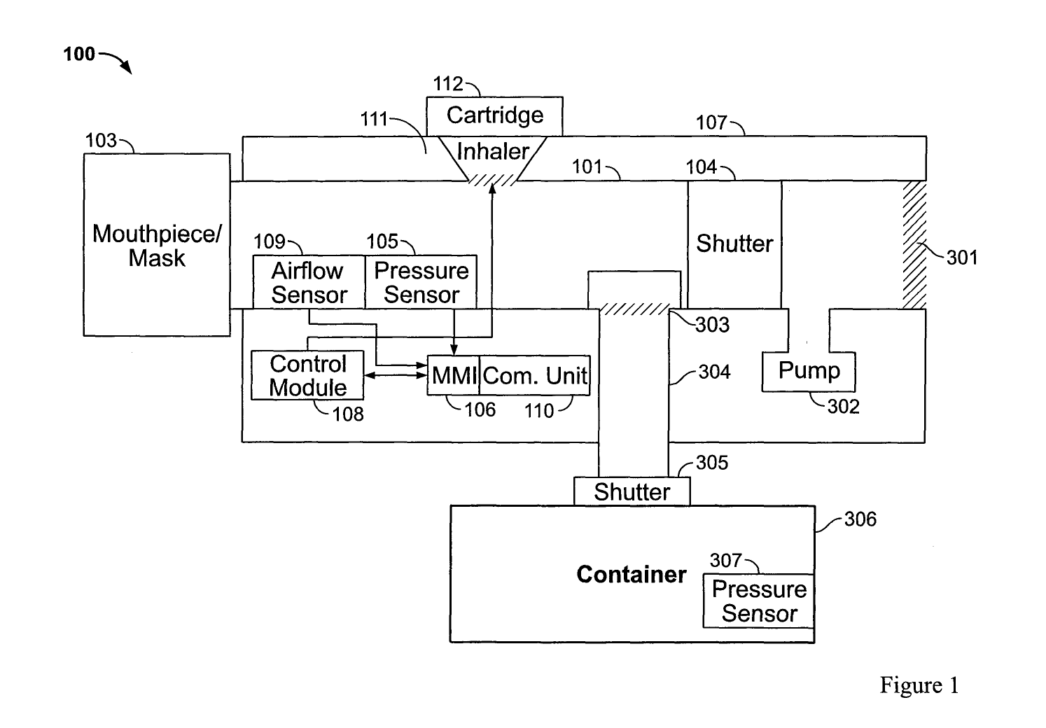

Reference is now made to FIG. 1, which is a schematic illustration of a breathing device 100 for performing controlled occlusion of airways. In some embodiments, the breathing device 100 may perform a rapid injection or extraction of air, also suitable for a Negative Expiratory Pressure (NEP) test, while measuring instantaneous alveolar pressure and/or volume. Breathing apparatus 100, in the illustrated embodiment, includes sensors 105, 307 and 109, a Man-Machine Interface (MMI) 106, a communication unit 110, a shutter 104, a shutter 305, a control module 108, a mouthpiece/mask 103 and an external container. Such components, may be substantially similar to corresponding components described in one or more of the included figures.

In some embodiments, for example, the control module 108 may be a computer or processing device such as, for example, a blade server, general-purpose personal computer (PC), Macintosh, workstation, Unix-based computer, or any other suitable device. In other words, the present disclosure contemplates computers other than general purpose computers as well as computers without conventional operating systems. In addition, the control module 108 may be a computer or processing device that is communicably coupled (e.g., through or wired or wireless connection or combination thereof) with another, remote computer or processing device, such as a general-purpose personal computer (PC), Macintosh, workstation, Unix-based computer, or any other suitable device. Thus, data and/or instructions sent to the control module 108, including instructions or commands provided to the control module 108 through the MMI 106 by a user of the device 100, may also be transmitted to the control module 108 from the remote computer or processing device. In addition, data provided to the MMI 106 from the control module 108 to display to the user may also be communicated to the remote computer for display, further processing, manipulation, or other purpose.

Control module 108 may be adapted to execute an operating system including Linux, UNIX, Windows Server, or any other suitable operating system. Further, the control module 108 may be communicably coupled to or include a local memory. The memory may include any memory or database module and may take the form of volatile or non-volatile memory including, without limitation, magnetic media, optical media, random access memory (RAM), read-only memory (ROM), removable media, or any other suitable local or remote memory component. The memory may include any data, software application, source code, objects, modules or other algorithms, as well as any other appropriate data such as VPN applications or services, firewall policies, a security or access log, print or other reporting files, HTML files or templates, data classes or object interfaces, child software applications or sub-systems, and others.

The breathing apparatus 100 illustrated in FIG. 1 also includes a closable flow tube 304 having a valve 303 that is connected to the chamber 101 between the shutter 104 and the mouthpiece 103. The illustrated breathing apparatus 100 also includes a ventilation valve 301 that is positioned in the backend of the chamber 101, and a pump 302 that is connected to the chamber 101 between the shutter 104 and the ventilation valve 301. In some aspects, the pump 302 is designed to propel air away from the chamber 101, thereby to induce expiration or resist inspiration. Optionally, the pump 302 may be designed to propel air into the chamber 101, and thereby induce inspiration or resist expiration. Optionally, the pump 302 may be designed to pump air in an oscillating manner thereby producing periodic movement of air in and out of chamber 101.

In some embodiments, the closable flow tube 304 may be a T shaped tube that allows the airflow in the breathing device 100 to bypass the shutter 104. Alternatively, the closable flow tube 304 may be a T shaped tube that directs airflow into a sealed container 306. The closable flow tube 304 may be closed automatically and/or manually at one or more velocities using shutter 305.

The pump 302, in some aspects, is located between the mouth of the user and the shutter 104. Alternatively, the shutter 104 is located between the mouth of the user and the pump 302. Optionally, the chamber 101 is connected to an external container of varying volumetric capacity. Optionally, the pump 302 injects or extracts air from container 306 or chamber 101 at a rate that is comparable to or significantly faster than the airflow rate of respiration at the instant of measurement. Optionally, pump 302 extracts or injects air to container 306 while shutter 305 is closed, thereby creating a positive or negative pressure difference between chamber 101 and container 306.

As illustrated, shutter 104 is constructed in the chamber 101, for example in a plane that is perpendicular to an axis between the ventilation end 102 and the mouthpiece end 103. In such a manner, the shutter 104 may regulate the passage of air flux in the chamber 101. The shutter 104 is connected to a control module 108 that is designed to control the opening and/or closing of the shutter 104 and shutter 305, for example as outlined above and described below. The control module 108 is optionally designed for receiving the outputs of the pressure sensor 105 and optionally a respiratory airflow sensor 109 and pressure sensor 307, for example as described below, and executing the exemplary methods herein. In some aspects, outputs of the control module 108 may be presented on the MMI 106. In some embodiments, the respiratory airflow sensor 109 and the pressure sensor 105 are the same sensor.

In some embodiments, the shutter 104 (and/or the shutter 305) may be a computer-activated Bosch solenoid valve (model 1147412020, Karlsruhe, Germany) modified in order to reduce airway resistance, dead-space and closing time. These modifications include, for example, reducing the airway resistance of the inlet and outlet of the valve, reducing the weight and resistance of the shutting mechanism, and increasing the solenoid gain. The modified valve has a dead space of 10 ml and flow resistance of 2 cmH₂O/L/s at a flow rate of 1 L/s. Upon activation, 80% of the occlusion is generated within the last 5 ms of solenoid travel, the valve fully closes within 10 ms, and remains closed for 80 ms. For flows ranging between ±1.5 L/s the valve closing time was found to be close to 10 ms. The leakage by the valve post closure was found to be <0.001 L/s.

The pressure sensor 105, in the illustrated configuration of the breathing device 100, is positioned between the shutter 104 and the mouthpiece end 103. The pressure sensor 307, in the illustrated configuration of the breathing device 100, is positioned between valve 303 and container 306, inclusive. The pressure sensors 105 and 307 may be any pressure measurement component, such as manometer or sensor for the measurement of absolute pressure with an analog to digital sampling rate of 100 Hz, 1000 Hz, 5000 Hz, 10,000 Hz or any intermediate value or larger value. In some embodiments, the pressure sensors 105 and 307 may be a pressure sensor as described in Honeywell Catalog #40PC001B1A. Alternatively, the pressure sensors 105 and 307 may be a pressure sensor such as a Samba 3000 pressure transducer, which is available from Linton Instrumentation of Norfolk, England. Of course, these are examples, and other pressure measuring sensors may be used for the pressure sensors 105 and 307.

The pressure sensors 105 and 307 may be fabricated for example from a respiratory airflow resistive means and a differential pressure manometer, or alternatively from a Pitot tube and a differential pressure manometer. The differential pressure manometer may be any suitable sensor with an analog to digital sampling rate of 100 Hz, 1000 Hz, 5000 Hz, 10,000 Hz or any intermediate value or larger value. Such differential pressure manometers may be similar or identical to the differential pressure manometer described at Honeywell Catalog #DC002NDR4.

In some aspects, a flow sensor 109, such as a mass respiratory airflow sensor, is positioned in the chamber 101. In the illustrated embodiment, the flow sensor 109 is positioned between the shutter 104 and the mouthpiece end 103. The flow sensor 109 may be any flow sensor, such as a hot wire mass respiratory airflow sensor. In some embodiments, the flow sensor 109 may be a Honeywell hotwire Mass Air Flow Sensor (model AWM 730B5, Golden Valley, Minn., US) with a working range of ±5 L/s. To calibrate this flow sensor, a 3 L syringe was used and cycled four times at widely varying flow rates, and then fitted these known volumetric changes to a multiparametric nonlinear function. To further validate the flow sensor and especially its dynamics, the volume container was instrumented with a pressure sensor (Endevco 8501-B1, San Juan Capistrano, Calif., US) which is linear and has a flat frequency response to 20 kHz. Gas flow from the container was then deduced during the interruption using Boyle's law. Fourier analysis of flow signals from the flow sensor relative to those deduced from chamber pressure by Boyle's law showed frequency

The container 306 is connected to the chamber 101 using the T tube 304. In some aspects, the container 306 may be made of a rigid or elastic material. In some aspects the container 306 is thermally insulated. In some aspects container 306 includes internal reinforcements such as crossbars and ribs which may prevent the container from expanding or contracting, damp material vibrations in the apparatus, or allow reducing overall weight of the apparatus. In some aspects the container is of cylindrically or spherically shaped so as to minimize shape deformation due to changing pressure in the container. In some aspects container 306 is an isothermal container. In some aspects container 306 is made isothermal by substantially filling the container with highly thermally conductive material, such as, for example, copper wool.

In the illustrated embodiment, the chamber 103 is constructed in a housing 107 generally sized and shaped so that it can be held comfortably in one hand, or mounted on a supporting frame, and held-up to a user's mouth while measuring the pulmonary alveolar pressure of the user, for example as outlined above and described below. The device 100 may also be configured so that the user can comfortably hold the device and self-monitor their respiratory parameters. The device 100 may also be stationary and configured so that the user's mouth is comfortably attached to the mask or mouthpiece 103 while the user is seated upright.

In some embodiments, the breathing device 100 includes the illustrated MMI 106. The MMI 106 may include a control panel, for example a keypad, a touch screen, and a set of buttons, and a liquid crystal display (LCD) screen. The keypad may include a start button, a selection button or other controls as desired to operate the breathing device 100, measuring pulmonary alveolar pressure of the user and or triggering shutter events. As illustrated, the MMI 106 may be communicably coupled to the control module 108 (which contains one or more processors or microprocessors for executing instructions stored on a tangible, computer readable media) to provide, for example, an interface for a user with the control module 108 and/or display results of processing performed by the control module 108. The control module 108 may also be communicably coupled to a memory (not shown) to store the measured pressure, the calculations which are based thereon, and/or instructions for execution on the aforementioned processors.

In a typical operation, the device 100 may be used for measuring a pulmonary alveolar pressure. For instance, pressure in the lungs is not uniform during normal respiration. A pressure gradient between the mouth and the lungs causes air to flow during normal respiration. During expiration, the airways' contraction and the elastic properties of the chest-wall, the diaphragm, and/or the alveoli may increase the pressure in the lungs above the pressure at the mouth (Pₘ), thereby inducing the expulsion of air from the lungs. During inspiration, inspiratory muscles cause the thoracic cage to lower the alveolar pressure below the pressure at the mouth, causing air to enter the lungs. As used herein, the term alveolar pressure (P_{Al}) means maximal and minimal pressure at the lungs during expiration and inspiration, respectively, at any given instant. In active lungs of a healthy user, for example during a continuous normal respiration, P_{Al} means a maximal pressure in the alveoli during expiration and minimal pressure in the alveoli during inspiration. In static lungs of a healthy user, P_{Al}=Pₘ. As used herein, ambient air refers to air at conditions surrounding the user and apparatus.

In some embodiments of the illustrated device 100, the shutter 104 and/or the chamber 101 may be adjusted to apply various resistance levels during an airway occlusion event. The difference between Pₘ and P_{Al} during breathing depends on the airway and device resistance. For example, the higher the airway and device resistance, the higher are Pₘ and P_{Al} amplitudes required to sustain a certain flow rate. In addition, for a given P_{Al}, Pₘ increases and the flow rate decreases with the incrementing of the device resistance. As the device resistance is configurable, the occlusion events may be changed. Optionally, the device resistance is different for expiration and inspiration. Additionally, in some embodiments, the device resistance may be dynamically changed (e.g., automatically by the control module 108). For example, the device resistance during a time interval prior to the occlusion event may be configured to be higher than the device resistance during an interval immediately after occlusion has ended. Such a configuration may aid in the reduction of Expiratory Flow Limitation prior to the occlusion event, thus reducing the time it takes alveolar and mouth pressure to equilibrate and improving the accuracy of the measured instantaneous volume. Such a configuration may also aid in inducing forced respiration or in stabilizing the respiration rate.

The variation of the resistances may be obtained by partially occluding airways by the shutter 104, for example, by changing a closure setting of the shutter 104 such that, at a particular setting, the shutter 104 partially occludes the airways in a closed state and, at another setting, the shutter 104 fully occludes the airways in the closed state. Optionally, the variation of the resistances may be obtained by allowing the user to manually change the caliber of the chamber 101 and/or the place of diffusive elements therein. Optionally, the variation of the resistances is obtained by allowing the user to use the MMI 106 for selecting a resistance.

In some embodiments of the present disclosure, the movement of one or more of the user's cheeks is limited for decreasing the responsiveness thereof to the airway occlusion events. Optionally, the limitation is performed by manually holding the cheeks, for example by instructing the user to hold her cheeks, by a caretaker, and/or automatically by a designated mask that is connected to the breathing device 100.

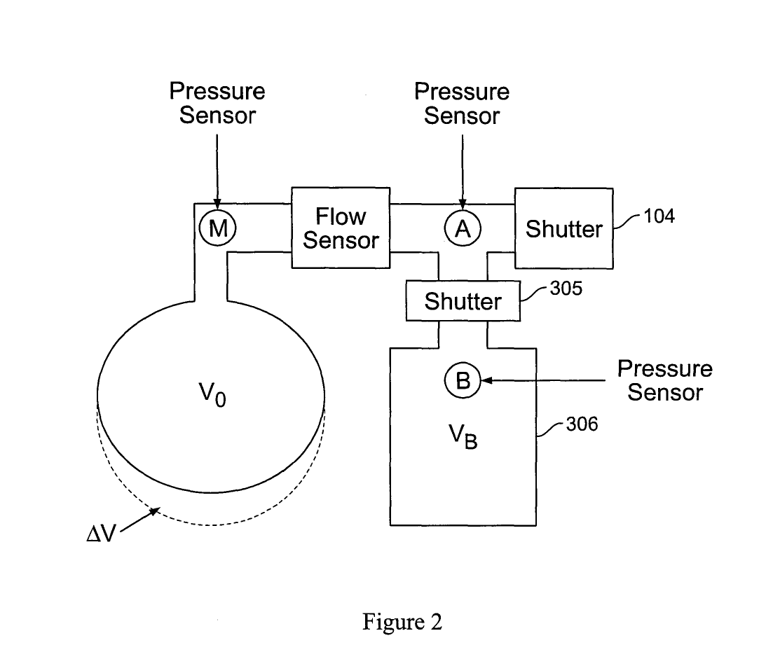

Turning to FIG. 2, this figure illustrates the user-device system composed of the interface of a user with one possible embodiment of breathing apparatus 100. The instantaneous volume of the respiratory system of the user at a particular instant is denoted by V₀. The volume of the rigid container is denoted as V_{B}. In some aspects, the volume of the container is of a volume similar to that of the respiratory system of the user. In some aspects, the volume of the container is larger than the volume of the respiratory system of the user, for example, 3 L, 5 L, 7 L, 10 L, 15 L, 20 L, 25 L, 30 L, or any intermediate value. Air enters and leaves the system through an outlet controlled by shutter 104. Air enters and leaves container through an outlet controlled by shutter 305. The rate of airflow is measured by airflow sensor 109, located between the mouth of the user and shutter 305. Pressure is measured at several possible locations: at the mouth by pressure sensor 105, in the vicinity of shutter 305 by pressure sensor 307, or inside container, by pressure sensor 307. Now, when shutter 104 and shutter 305 are open, changes in the lung volume of the user produce changes in air density, resulting in changes of the user-device system pressure, and airflow in and out of the user-device system. When shutter 104 is closed and shutter 305 is open, changes in the lung volume of the user result in changes of the user-device system mean pressure and density, and exchange of air between the lungs of the user and container.

In some aspects, if shutter 305 is at its closed state, the user-device system may operate as a common interrupter device, with appropriate uses. For example, in some aspects, the shutter 305 may be configured to operate quietly so as not to create any reflexes or undesired responses by the subject, thereby avoiding inaccuracies of measurement. More importantly, shutter 305 may be configured to operate quickly, both in terms of its shutting speed (i.e., the time it takes for the shutter to go from an open state to a closed state and vice versa) and in terms of its shutting duration (i.e., the period of time for which the shutter is closed). The shutting speed is in some embodiments less than 10 ms, preferably less than 5 ms, and more preferably less than 2 ms. The shutting duration is in some embodiments less than 2 seconds and preferably less than 100 ms. This fast paced shutting speed and shutting duration may provide more accurate and reliable measurements of TGV, TLC and RV. The high speed operation of shutter 305 and high rate of data acquisition may result from the typical response time of the lungs to abrupt occlusion of the airways while breathing. The response time of the lungs of a human being is in the order of ms to tens of ms, and accurate recording of the details of the response of the lungs to such abrupt occlusion is essential for accurate calculation of the internal volume of the lungs.

Now, if shutter 104 is open and shutter 305 is closed, during normal breathing air is exchanged only between the respiratory system and atmospheric air. Suppose now that pump 302 extracts a portion of air molecules from container 306 such that the pressure in container 306, optionally read by pressure sensor 307, is lower than atmospheric pressure (e.g., an instantaneous Negative Expiratory Pressure). Now, during normal expiration of air by the user, when shutter 305 is suddenly opened, an increased pressure difference between the container 306 and lungs may produce a force acting to accelerate the expulsion of air by the user. Airflow of subjects suffering from expiratory flow limitation, however, is limited due to the collapse of airways beyond a certain airflow rate. Thus, by applying instantaneous Negative Expiratory Pressure (NEP) during normal or forced breathing, the level of flow limitation of the user may be estimated by correlating it with the increase in airflow rate for a given negative pressure difference. Such tests, which may be performed by the breathing device 100 are known in the art as NEP testing. Breathing device 100, therefore, may be used to perform standard spirometric testing, lung volume measurement as described below, and NEP testing during one continuous measurement or separate measurements.

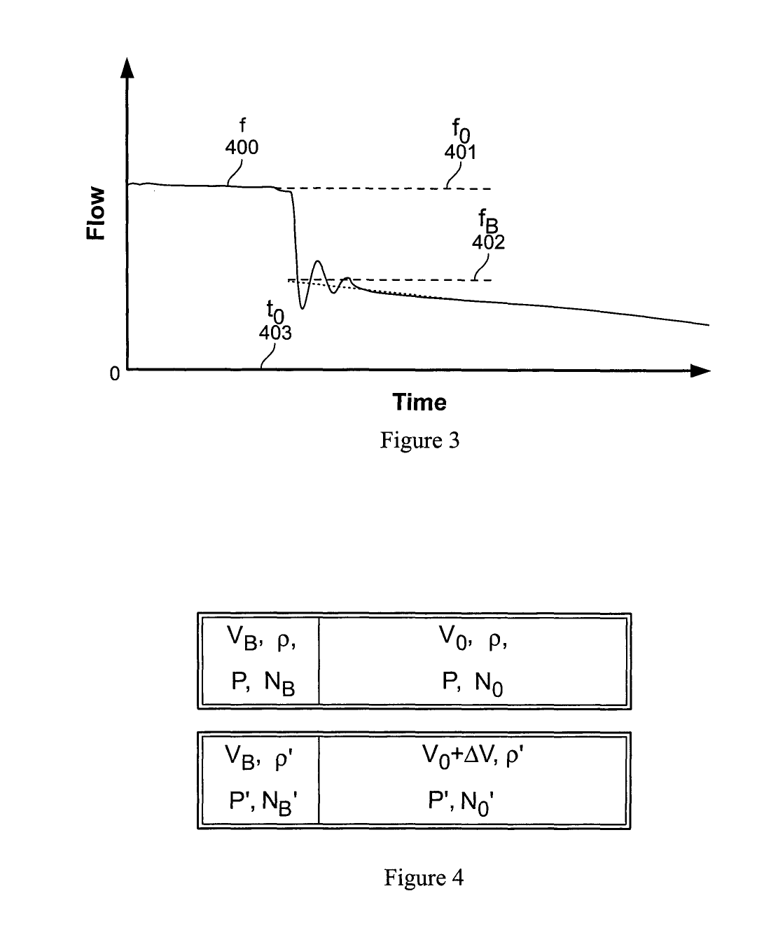

Turning to FIG. 3, this figure illustrates a graph 400 that schematically depicts a time dependence of airflow recorded by airflow sensor 109. Prior to instant to 403, the air flow rate is substantially equal to the volumetric rate of change of the user's lungs, as shown above in reference to FIG. 2 where shutter 104 and shutter 305 are open. At the instant t₀ 403, shutter 104 occludes airways. At instants after t₀ 403, the flow recorded by sensor 109 is equal to the rate of exchange of airflow between container and the lungs.

Turning briefly to FIG. 4, this figure illustrates two states of a sealed container containing ideal gas, partitioned into two volumes, partition-0 and partition-B, of volumes V₀ and V_{B}, respectively, that are in thermodynamic equilibrium. The volume of partition-0 is then changed while keeping the volume of partition-B constant. The air density in the sealed container prior and after the volume change, ΔV is denoted by ρ and ρ′, respectively. The pressure in the sealed container prior and after the volume change is denoted by P and P′, respectively. The number of gas molecules in partition-0 and in partition-B prior and after the volume change are denoted by N₀ and N_{B} and N₀′ and N_{B}′, respectively. The number of molecules being exchanged between partition-0 and partition-B during the volume change is denoted as ΔN.

By using conservation of mass or the ideal gas law, for a relatively small change in volume (i.e., ΔN<<N_{B}) the change in volume is approximately related to the above quantities by:

$$Δ V = - Δ N \frac{( {V}_{B} + {V}_{0} )}{{N}_{B}}$$

Now, if the density of air has not substantially changed from its base value during the volume change, the above equation can be written as

$$Δ V = Δ {V}^{′} \frac{{V}_{0} + {V}_{B}}{{V}_{B}}$$

where ΔV′ denotes the number of molecules exchanged between partition-0 and partition-B divided by ρ, the base molecule number density of air. Now, for small changes in volume, dividing both sides of the above equation by small time intervals, Δt, the above equation can be written as

$$\frac{Δ V}{Δ {V}^{′}} = \frac{ \frac{Δ V}{Δ t}}{\frac{Δ {V}^{′}}{Δ t}} = \frac{f}{{f}^{′}} = \frac{{V}_{0} + {V}_{B}}{{V}_{B}}$$

where f denotes the volumetric rate of change of the system, and f′ the proportionate rate of exchange of volume between partition-0 and partition-B. The above equation can also be written as

$${V}_{0} = \frac{{V}_{B}}{γ} \frac{f - {f}^{′}}{{f}^{′}}$$

where γ is a gas constant.

The above equation for calculating instantaneous lung volume may be valid if both container 306 and the respiratory system are at isothermal or adiabatic conditions. The human respiratory system is generally considered to be isothermal. Isothermal conditions at container 306 may be obtained by, for example, inserting copper wool or some other thermally conductive material into container 306 at a density that ensure that heat transfer to the highly conductive material can be obtained at a rate of 10 Joule per second, or higher. Approximate adiabatic conditions may be achieved at the respiratory system of the subject and container for very fast volume changes (several ms) during which heat flow out of the gas of the container and respiratory system may be considered negligible. If both V₀ (i.e., the respiratory system of the user) and container are either at adiabatic or isothermal conditions, the gas constant γ is 1. If V₀ denotes the lung volume at isothermal conditions and V_{B} a container at adiabatic conditions, for gas at normal atmospheric conditions γ=7/5. If homogeneous isothermal or adiabatic conditions are not kept at the respiratory system of the subject and rigid container 306 system, second order corrections may be introduced to the above equations for calculating instantaneous lung volume without affecting the generality of the method. For example, if conditions at the container and/or respiratory system of the user are only partially adiabatic or isothermal, the gas constant γ may be set to a value in the range 1<γ<7/5, depending on the specification of the device or measurement.

Returning to FIG. 3, the above equation is used to calculate V₀ by equating f with the rate of airflow recorded prior to t₀ and f′ with the flow recorded after t₀. Now, due to the inertia of the respiratory system of the user, it may be assumed that for sufficiently fast occlusion of airways, the volumetric rate of change of the respiratory system of the subject immediately prior to the occlusion of airways by shutter 104, denoted as f₀, remains constant shortly after. The instantaneous volume of the respiratory system of the subject at t₀ is then calculated by

$${V}_{0} = {V}_{B} ( \frac{{f}_{0}}{{f}_{B}} - 1 ) \qquad ( 1 )$$

where f_{B} denotes the recorded airflow rate immediately after shutter 104 has occluded airways at t₀. In the case of non-ideal shutting of shutter 104, the flow signal after t₀ can be back extrapolated in order to determine f_{B} as shown in FIG. 3. Optionally, flow level f_{B} may be obtained by averaging the flow signal over some range after t₀, for example, from 1 ms after t₀ to 3 ms after t₀, from 5 ms after t₀ to 10 ms after t₀, from 2 ms after t₀ to 15 ms after t₀, or any such range between 0 to 20 ms after t₀.

The above equation for calculating the instantaneous volume may be valid under quasi-static conditions, which are obtained at relatively low flows. As the rate of flow, f₀, increases, density gradient in the respiratory system and the rigid container may result in a loss of accuracy of the measurement. Such second order corrections to the above formula may be introduced, without affecting the generality of the method. For example, a combination of the pressure recordings at several locations in the system, for example, at point m and point B, as shown in FIG. 2, may be used to measure the density and pressure gradients in the system and introduce second order corrections to the above equations for the calculation of instantaneous lung volume, involving the inhomogeneous distribution of pressure and density of the system.

Optionally, the airflow rate of change after t₀ may be used to estimate the rate of change of lung volume, and added to the right hand side of the above equation as a linear second order correction, such that

$${V}_{0} = {V}_{B} ( \frac{{f}_{0}}{{f}_{B}} - 1 ) + {X}_{i} {Y}_{i} \qquad ( 2 )$$

where Xᵢ is some coefficient and Yᵢ is the rate of change of airflow shortly after t₀. Similarly, Xᵢ and Yᵢ may be used to denote any set of linear correction coefficients and parameters, respectively, which may also include the rate the of change of airflow prior to the interruption, device resistance, airway resistance, airway compliance, an index of deviation from adiabatic or isothermal conditions, or any parameter which may produce a deviation from the prediction of the idealized equation for V₀ (Eq. 1). Optionally, Yᵢ is a parameter derived using some reading of pressure sensor 105, or pressure sensor 307, for example, the pressure level immediately after t₀ or some averaged pressure level shortly after t₀. Similarly, V_{B} may be set to a value different than the volume of the container due to non-ideal adiabatic or isothermal conditions of the respiratory system of the user and/or container. Similarly, the term XᵢYᵢ may denote a constant offset parameter.

The time interval during which airways remain occluded by shutter 104 will be termed here as occlusion duration. The minimal occlusion duration is a time interval that would allow for an accurate determination of f_{B}. Such occlusion durations range between 10 ms to 100 ms, for example 20 ms, 30 ms, 50 ms, 70 ms, 90 ms, or any intermediate value. The time interval it takes shutter 104 to achieve substantial occlusion of airways will be termed here as shutting duration.

A substantial occlusion of airways is defined here as reduction of the cross-sectional area of the shutter by 90% or more. The shutting duration of shutter 104 may be fast enough for changes in the rate of change of lung volume to be negligible between the instant at which f₀ is determined and the instant at which f_{B} is determined. Such shutting durations range between 0.5 ms and 30 ms, for example 1 ms, 5 ms, 10 ms, 15 ms, 20 ms, 25 ms, or any intermediate value. After shutter 104 reopens, the user may continue breathing normally.

Optionally, shutter 104 is designed to have a minimal effect on the response of the lungs to the occlusion, for example by inducing a minimal amount of mechanical work thereon. Optionally, the occlusion is imperceptive to the user undergoing the measurement so as to assure that no respiratory change that might affect the normal rate of flow during the occlusion is aroused by the occlusion shutter action. Optionally, the occlusion is performed at any stage of the users breathing cycle, for example during early inspiration, late inspiration, early expiration, late expiration, or any intermediate stage.

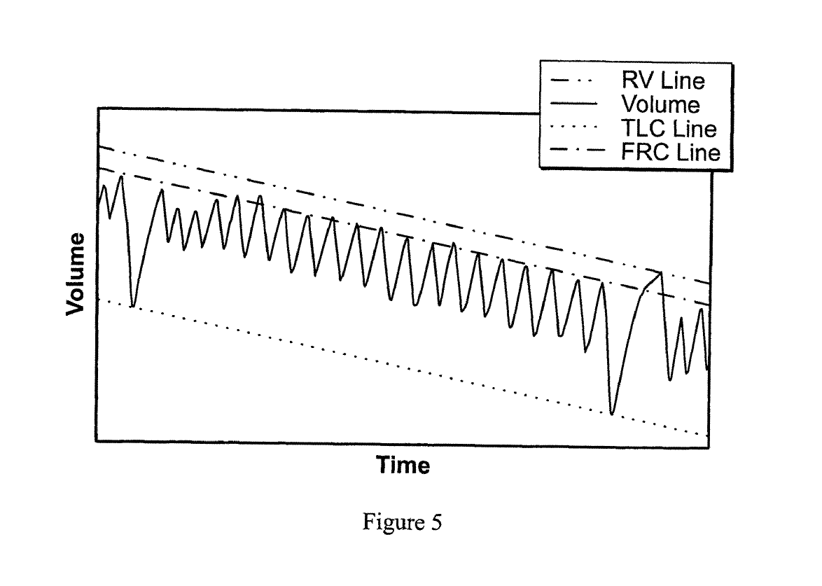

The TLC, RV or TGV level of the user may be calculated by referencing the calculated instantaneous volume with points of maximal inhalation, maximal exhalation or maximal exhalation during continuous tidal breathing, respectively. For example, FIG. 5 depicts a continuous recording of pulmonary volume change pertaining to a user. The breathing pattern causes inflating and/or deflating of the lungs so that their volume is changed with respect to the various repetitive respiratory stages. In FIG. 5, the breathing pattern is such that the user reaches the TLC level at the beginning of the measurement, then breathes tidally and ends with a vital capacity (VC) breathing maneuver by inhaling to the TLC level and exhaling slowly all of the available Vital Capacity of the lungs down to the RV level. In such a breathing pattern, the user reaches the TGV several times and the TLC level at the beginning and the end of the measurement. This breathing pattern allows for determining the airflow that is contributed by the drift and reducing it from the respective instantaneous pulmonary volume.

Drifts may result from a sensor bias or leakage. However, under properly functioning condition, the major contribution to the drift is caused by body-room temperature and saturation differences, which may vary from patient to patient according to the subject's pulmonary properties and breathing pattern. Such drifts may reach up to +/−3 Liters per minute in normal users and in patients suffering from pulmonary disorders. Failure to correct for the drift may result in a substantial loss of accuracy which may render the result of the measurement as diagnostically irrelevant or even harmful in the case of misdiagnosis. The occlusion of airways may be performed several times during the continuous recording of lung volume changes and the resultant calculated TLC, RV or FRC of the user may be outputted as a statistically averaged result of some of the occlusion events.

Optionally, shutter 104 is designed to re-open fast enough for changes in the rate of change of lung volume to be negligible between the instant at which an airflow level immediately after the occlusion is determined and the instant at which an airflow level immediately prior to the re-opening of the shutter is determined. Such shutter opening durations range between 0.5 ms and 30 ms, for example 1 ms, 5 ms, 10 ms, 15 ms, 20 ms, 25 ms, or any intermediate value. The above formula can be used to calculate instantaneous lung volume by equating f₀ with an airflow level immediately after the shutter reopens and equating f_{B} with an airflow level immediately prior to the shutter reopening.

Optionally, airflow rate after t₀ is deduced from the rate of change of pressure in container 306, measured by pressure sensor 307 or pressure sensor 105 by using

$$f = \frac{{V}_{B}}{{P}_{A} γ} \frac{d P}{d t}$$

where f denotes airflow rate, P_{A} denotes atmospheric pressure, dP/dt denotes the rate of change of the pressure in the container after t₀, and γ denotes the adiabatic constant which equals 1 for an isothermal container and 1.4 for an adiabatic container.

Returning briefly to FIG. 2, a method for the measurement of instantaneous lung volume is now described involving the sequential timing of shutter 104 and shutter 305. In particular, a method is now described in which shutter 104 occludes airways at a first instant and shutter 305 occludes container 306 at a second instant following the first instant.

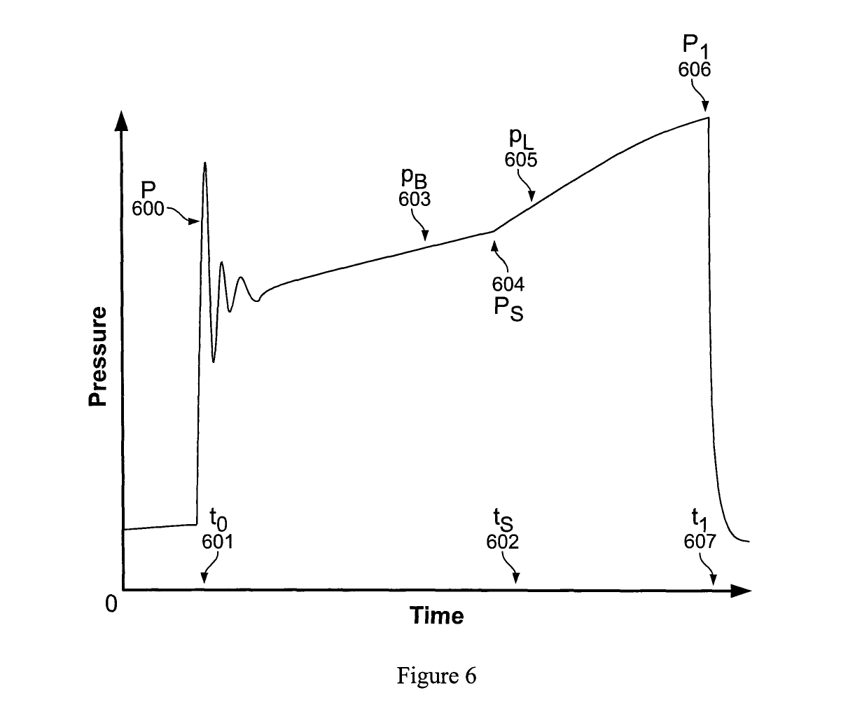

Turning to FIG. 6, a typical gauge pressure reading (i.e., pressure change from ambient pressure) over time, 600, during a sequential occlusion event is shown. At instant t₀ 601, shutter 104 occludes airways. Pressure in the system composed of the user's lungs and container is first equilibrated shortly after the occlusion and then due to the inertia of the respiratory system or muscle work, pressure deviates from base alveolar pressure. At instant tₛ 602, shutter 305 occludes airways connecting the user's respiratory system and container. The pressure slope shortly prior to tₛ, reflecting the rate of change of pressure in the system over time is denoted as p_{B}, 603. The rate of change of pressure in the user's respiratory system shortly after tₛ, is denoted as p_{L}, 605. The pressure value at the instant tₛ is denoted as P_{S}, 604. At instant t₁ 607 shutter 104 reopens and the user may continue to breathe normally.

Now, if container is kept at isothermal conditions, as described above, during the time interval between t₀ and tₛ the system composed of the user's respiratory system and the rigid container may obey Boyle's law, written as

$${V}_{0} + {V}_{B} = ( {P}_{A} + Δ P ) \frac{Δ V}{Δ P}$$

where ΔV denotes changes in volume, ΔP denotes changes in pressure, P_{A} denotes ambient pressure, V₀ denotes instantaneous respiratory system volume and V_{B} denotes the volume of the rigid container. Now, for short enough time intervals, Δt, the term ΔV/ΔP may be replaced with

$$\frac{Δ V}{Δ P} = \frac{ \frac{Δ V}{Δ t}}{\frac{Δ P}{Δ t}} = \frac{f}{p}$$

where ΔV and ΔP denote the changes in volume and pressure, respectively, during the interval Δt, f denotes the volumetric rate of change with respect to time and p denotes the pressure rate of change with respect to time. Shortly prior to tₛ, Boyle's law can be written as

$${V}_{0} + {V}_{B} = ( {P}_{A} + {P}_{S} ) \frac{{f}_{S}}{{p}_{B}}$$

where f_{S} denotes the volumetric rate of change at t_{S}.

Now, in embodiments with the container 306 and the respiration device 100 as rigid, all volumetric rate of change can be attributed to the respiratory system. Therefore, shortly after tₛ, it may be assumed that the volumetric rate of change, f_{S}, has not changed due to the occlusion of container 306 by shutter 305. Boyle's law for the respiratory system shortly after t_{S} can therefore be written as

$${V}_{0} = ( {P}_{A} + {P}_{S} ) \frac{{f}_{S}}{{p}_{L}}$$

The two above equations have two unknowns, V₀ and f_{S}. Using standard algebra V₀ is solved for by any person skilled in algebra,

$${V}_{0} = {V}_{B} \frac{{p}_{B}}{{p}_{L} - {p}_{B}}$$

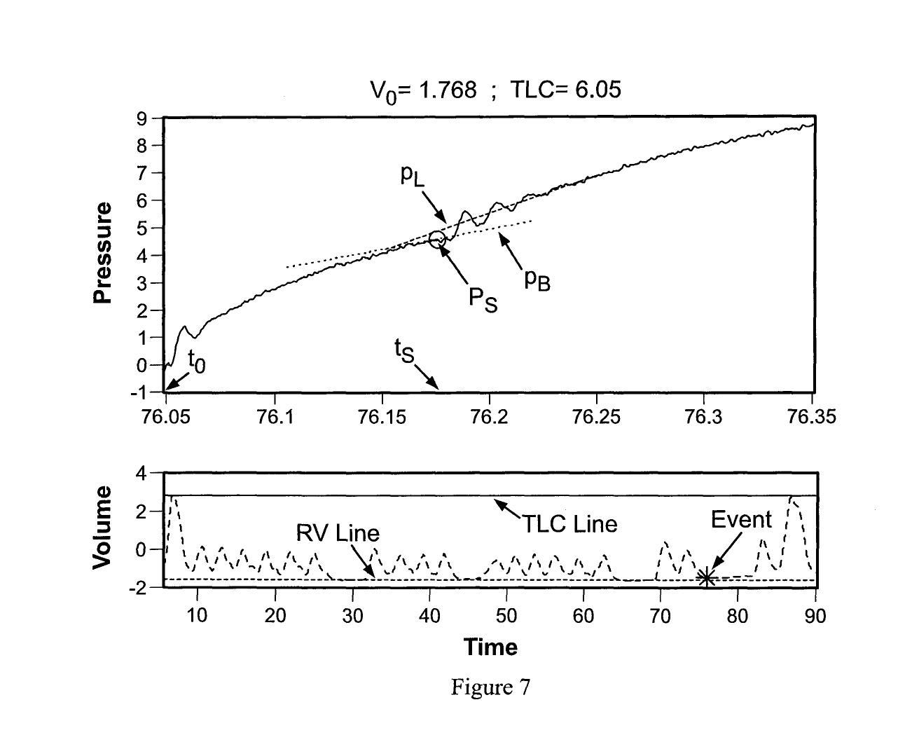

An example of a real event is shown in FIG. 7. The lower panel shows the dependence of respiratory system volume changes over time. Shortly after the beginning of the recording and shortly prior to the termination of the recording the user inhales up to the TLC level. A line connecting these two levels is denoted as the TLC line. The user exhales several times during the recording down to the RV level. A line marking the RV level is denoted as the RV line. The exemplary event is performed 76 seconds after the beginning of the recording. The upper panel shows a pressure curve, recorded at the mouth of the user, during the occlusion event. The instant at which shutter 104 occludes airways is denoted by t₀ in the upper panel. The instant at which shutter 305 occludes container 306 is denoted by t_{S} in the upper panel. The pressure level at t_{S} is marked by a circle and denoted by P_{S}. A line showing the approximated rate of change of the pressure with respect to time shortly prior to tₛ is shown by a dashed line, denoted as p_{B} in the upper panel. A line showing the approximated rate of change of the pressure with respect to time shortly after t_{S} is shown by a dotted line, denoted as p_{L} in the upper panel. The instantaneous volume, V₀, and the TLC of the user are calculated as described above and the result is displayed at the title of the upper panel.

Optionally, shutter 104 and shutter 305 are designed to have a minimal effect on the response of the lungs to the occlusion, for example by inducing a minimal amount of mechanical work thereon. Optionally, the occlusion may be imperceptive to the user undergoing the measurement so as to assure that no respiratory change that might affect the normal rate of flow during the occlusion is aroused by the occlusion shutter action. Optionally, the occlusion is performed at any stage of the user's breathing cycle, for example during early inspiration, late inspiration, early expiration, late expiration, or any intermediate stage. Optionally, shutter 305 occludes container significantly after shutter 104 has occluded airways in order to ensure that pressure gradient between lungs and mouth is minimal.

The above equation for calculating instantaneous lung volume using a sequential activation of shutter 104 and shutter 305 may be valid if the lungs and container 306 are at isothermal or adiabatic conditions, as described above. If homogeneous isothermal or adiabatic conditions are not kept at the lungs plus rigid container 306, second order corrections may be introduced to the above equations for calculating instantaneous lung volume without affecting the generality of the method. For example, if the respiratory system of the subject is assumed to maintain isothermal conditions, while container 306 is assumed to maintain adiabatic conditions, Boyle's law for the system during the time interval between t₀ and t_{S} may be written as

$${V}_{0} + {V}_{B} = \overline{γ} ( {P}_{A} + {P}_{S} ) \frac{{f}_{S}}{{p}_{B}}$$

where γ̅ is equal to

$$\overline{γ} = \frac{{V}_{0} + γ {V}_{B}}{{V}_{0} + {V}_{B}}$$

where γ is the adiabatic gas constant, equal to 1.4 for diatomic gases. As described above, V₀ can be extracted from the simple set of two equations for the two unknowns, V₀ and f_{S}, by requiring that V₀ be positive and choosing the appropriate root of the resulting quadratic equation.

The above equation for calculating the instantaneous volume may be valid under quasi-static conditions, which are obtained at relatively low flows. As the rate of flow, f₀, increases, density gradient in the respiratory system and the rigid container may result in a loss of accuracy of the measurement. Such second order corrections to the above formula may be introduced, without affecting the generality of the method.

The pressure recording shown on FIG. 7 and depicted on FIG. 6 may be recorded by a pressure sensor located at point B, shown on FIG. 2, inside container 306, at point m, at the users mouth, or point A, in the vicinity of shutter 104, and T-tube 304, or any combination thereof. In addition, a combination of the pressure recordings at several locations in the system, for example, at point m and point B, as shown in FIG. 2, may be used to measure the density and pressure gradients in the system and introduce second order corrections to the above equations for the calculation of instantaneous lung volume, involving the inhomogeneous distribution of pressure and density of the system.

Optionally, the rate of change of airway pressure, for example P_{S} or P_{B}, shown on FIG. 7, may be produced by the inertial motion of tissue involved in respiration, by the forcing of respiratory muscles, or by an external pump or a device in fluid communication of which can produce controlled volumetric changes.

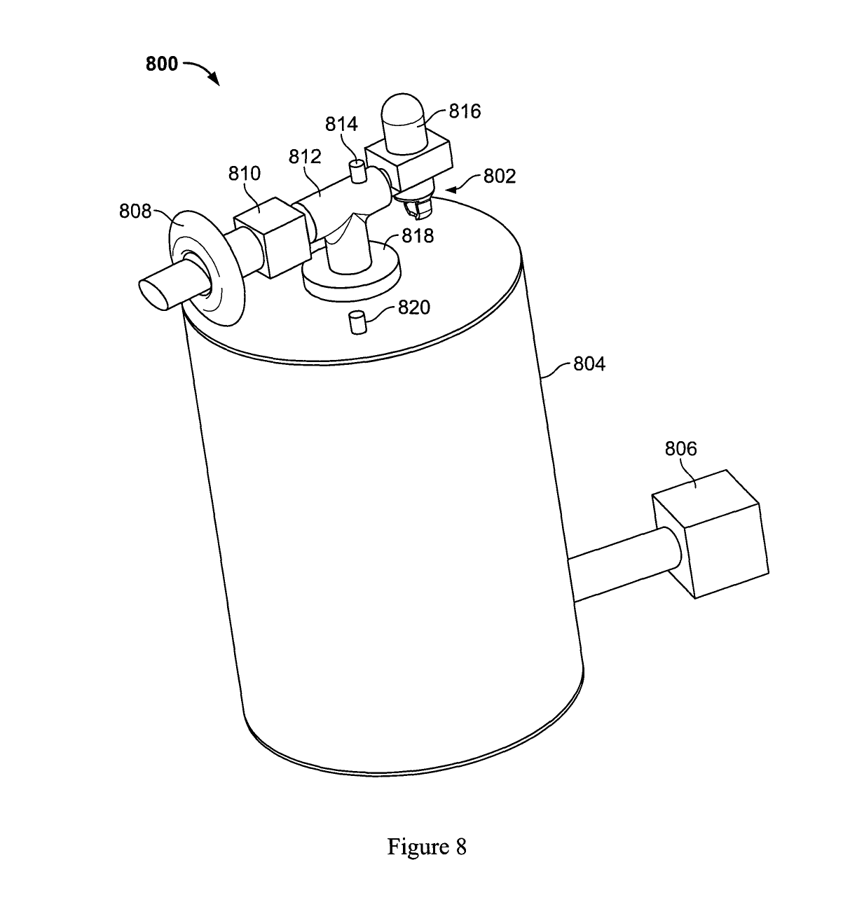

FIG. 8 is an illustration of an example breathing device 800 for performing controlled occlusion of airways. In some embodiments, the breathing device 800 (like the breathing device 100) may perform a rapid injection or extraction of air, also suitable for a Negative Expiratory Pressure (NEP) test, while measuring instantaneous alveolar pressure and/or volume. Breathing apparatus 800, in the illustrated embodiment, includes a breathing assembly 802 coupled to and in fluid communication with a container 804, which in turn is coupled to and in fluid communication with a pump 806. As illustrated, the breathing assembly 802 includes a mouthpiece 808, a flow sensor 810, a chamber 812, a pressure sensor 814, a shutter 816, and a shutter 818.

In some aspects, if shutter 818 is at its closed state, the breathing device 800 may operate as a common interrupter device, with appropriate uses. For example, in some aspects, the shutter 818 may be configured to operate quietly so as not to create any reflexes or undesired responses by the subject, thereby avoiding inaccuracies of measurement. More importantly, shutter 818 may be configured to operate quickly, both in terms of its shutting speed (i.e., the time it takes for the shutter to go from an open state to a closed state and vice versa) and in terms of its shutting duration (i.e., the period of time for which the shutter is closed). The shutting speed is in some embodiments less than 10 ms, preferably less than 5 ms, and more preferably less than 2 ms. The shutting duration is in some embodiments less than 2 seconds and preferably less than 100 ms. This fast paced shutting speed and shutting duration may provide more accurate and reliable measurements of TGV, TLC and RV. The high speed operation of shutter 818 and high rate of data acquisition may result from the typical response time of the lungs to abrupt occlusion of the airways while breathing. The response time of the lungs of a human being is in the order of ms to tens of ms, and accurate recording of the details of the response of the lungs to such abrupt occlusion is essential for accurate calculation of the internal volume of the lungs.

As illustrated, a pressure sensor 820 is mounted on a top portion of the container 804. In some embodiments, the breathing device 800 may also include a user interface (e.g., such as the MMI 106) and a control module (e.g., such as control module 108), which are not shown in FIG. 8.

As illustrated, the mouthpiece 808 facilitates fluid communication between an airways (e.g., lungs) of a subject and the chamber 812 and/or container 804. For example, in some embodiments, the mouthpiece 808 may limit movement of the subject's cheeks, thereby decreasing the responsiveness thereof to the airway occlusion events.

As illustrated, shutter 816 is constructed at an end of the breathing assembly 802 opposite the mouthpiece 808. Between the shutter 816 and the mouthpiece 808, and in fluid communication with the shutter 816 and the mouthpiece 808, are the flow sensor 810 and the chamber 812. In such a manner, the shutter 816 may regulate the passage of air flux in the chamber 812. The shutter 816 may be communicably coupled to a control module (not shown) that is designed to control the opening and/or closing of the shutter 816, as well as the shutter 305.

The pressure sensor 814, in the illustrated configuration of the breathing device 800, is positioned between the shutter 816 and the mouthpiece 808 and within the chamber 812. The pressure sensor 820, in the illustrated configuration of the breathing device 800, is positioned to measure pressure within the container 804, e.g., on an opposite side of the shutter 818 compared to the chamber 812. The pressure sensors 814 and 820 may be any pressure measurement component, such as manometer or sensor for the measurement of absolute pressure. The pressure sensors 814 and 820 may be fabricated for example from a respiratory airflow resistive means and a differential pressure manometer, or alternatively from a Pitot tube and a differential pressure manometer.

The flow sensor 810, such as a mass respiratory airflow sensor, is positioned between the mouthpiece 808 and the chamber 812. The flow sensor 810 may be any flow sensor, such as a hot wire mass respiratory airflow sensor. In some embodiments, the respiratory airflow sensor 810 and the pressure sensor 814 may be combined in a single sensor.

The container 804 is connected to the chamber 812 with a T tube and the shutter 818 is positioned between the T tube and container 804. In some aspects, the container 804 may be made of a rigid or elastic material. In some aspects the container 804 is thermally insulated. In some aspects container 804 is an isothermal container, such as, by filling the container 804 with highly thermally conductive material, for example, copper wool.

As illustrated, the T tube may be closed to fluid communication between the chamber 812 and the container 804 by the shutter 818 (e.g., automatically and/or manually). In some embodiments, the pump 806 extracts or injects air into container 804 while the shutter 818 is closed, thereby creating a positive or negative pressure difference between the chamber 812 and the container 804.

The pump 806 may propel air away from the chamber 812, thereby to induce expiration or resist inspiration in the subject (e.g., through the mouthpiece 808). Optionally, the pump 806 may propel air into the chamber 812, and thereby induce inspiration or resist expiration in the subject. Optionally, the pump 806 may be designed to pump air in an oscillating manner thereby producing periodic movement of air in and out of chamber 812.

In a typical operation, the device 800 may be used for measuring a pulmonary alveolar pressure. For instance, pressure in the lungs is not uniform during normal respiration. A pressure gradient between the mouth and the lungs causes air to flow during normal respiration. During expiration, the airways' contraction and the elastic properties of the chest-wall, the diaphragm, and/or the alveoli may increase the pressure in the lungs above the pressure at the mouth (Pₘ), thereby inducing the expulsion of air from the lungs. During inspiration, inspiratory muscles cause the thoracic cage to lower the alveolar pressure below the pressure at the mouth, causing air to enter the lungs. As used herein, the term alveolar pressure (P_{Al}) means maximal and minimal pressure at the lungs during expiration and inspiration, respectively, at any given instant. In active lungs of a healthy user, for example during a continuous normal respiration, P_{Al} means a maximal pressure in the alveoli during expiration and minimal pressure in the alveoli during inspiration. In static lungs of a healthy user, P_{Al}=Pₘ.

In some embodiments of the illustrated device 800, the shutter 816 and/or the chamber 812 may be adjusted to apply various airway resistance levels during an airway occlusion event. The difference between Pₘ and P_{Al} during breathing depends on the airway and device resistance. For example, the higher the airway and device resistance, the higher are Pₘ and P_{Al} amplitudes required to sustain a certain flow rate. In addition, for a given P_{Al}, Pₘ increases and the flow rate decreases with the incrementing of the device resistance. As the device resistance is configurable, the occlusion events may be changed. Optionally, the device resistance is different for expiration and inspiration. Additionally, in some embodiments, the device airway resistance may be dynamically changed (e.g., automatically by a control module connected to the breathing device 800). For example, the device resistance during a time interval prior to the occlusion event may be configured to be higher than the device resistance during an interval immediately after occlusion has ended. Such a configuration may aid in the reduction of Expiratory Flow Limitation prior to the occlusion event, thus reducing the time it takes alveolar and mouth pressure to equilibrate and improving the accuracy of the measured instantaneous volume. Such a configuration may also aid in inducing forced respiration or in stabilizing the respiration rate.

The variation of the resistances may be obtained by partially occluding airways by the shutter 816, for example, by changing a closure setting of the shutter 816 such that, at a particular setting, the shutter 816 partially occludes the airways in a closed state and, at another setting, the shutter 816 fully occludes the airways in the closed state. Optionally, the variation of the resistances may be obtained by allowing the user to manually change the caliber of the chamber 816 and/or the place of diffusive elements therein.

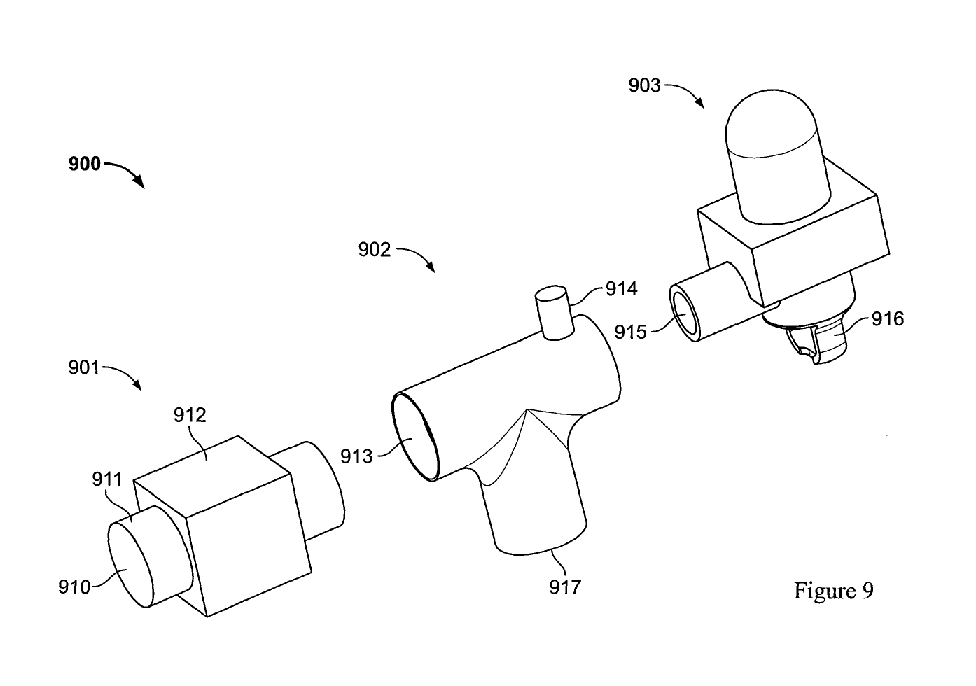

Referring to FIG. 9, this figure is an illustration of an example breathing assembly 900 of a breathing device (such as the breathing device 100, breathing device 800, or other breathing device according to the present disclosure) for performing controlled occlusion of airways. As illustrated, the breathing assembly 900 includes a flow sensor 901, an airways chamber 902, a shutter assembly 903 including a ventilation valve 916, and a pressure sensor 914. In some aspects, these components are substantially similar and in some cases, identical, to similarly-named components described above with respect to the breathing device 100 and/or the breathing device 800.

FIG. 9, for example, illustrates example relative positions of the flow and pressure sensors, valves, and design of airways of the breathing assembly 900. For example, an inner diameter of a flow tube 912 (e.g., in which the flow sensor may be placed) and a flow tube 913 (e.g., in which the flow sensor may be placed), may be 10 mm, 12 mm 15.3 mm, 17 mm, 20 mm, 25 mm, 28 mm, 30 mm, 33 mm, 40 mm or any intermediate value. An external diameter 911 of the flow tube 912 may be 15 mm, 25 mm, 28 mm, 30 mm, 33 mm, 40 mm or any intermediate value. A valve-T-tube interface 915 may be made of elastic material or shock absorbing material to facilitate shock and vibration absorbance as well as improved sealing. In some aspects the airway resistance at the airway resistance of an outlet 917, connecting the airway T-Tube 902 with an external container, has the same airway resistance as the shutter assembly 903, for example, 10 cmH₂O/L/s at a flow rate of 1 L/s, 5 cmH₂O/L/s at a flow rate of 1 L/s, 2 cmH₂O/L/s at a flow rate of 1 L/s, 1 cmH₂O/L/s at a flow rate of 1 L/s, 0.1 cmH₂O/L/s at a flow rate of 1 L/s, or any intermediate value, so as to avoid a change in the airflow rate at the mouth of the user due to a change in airway resistance of breathing assembly 900.

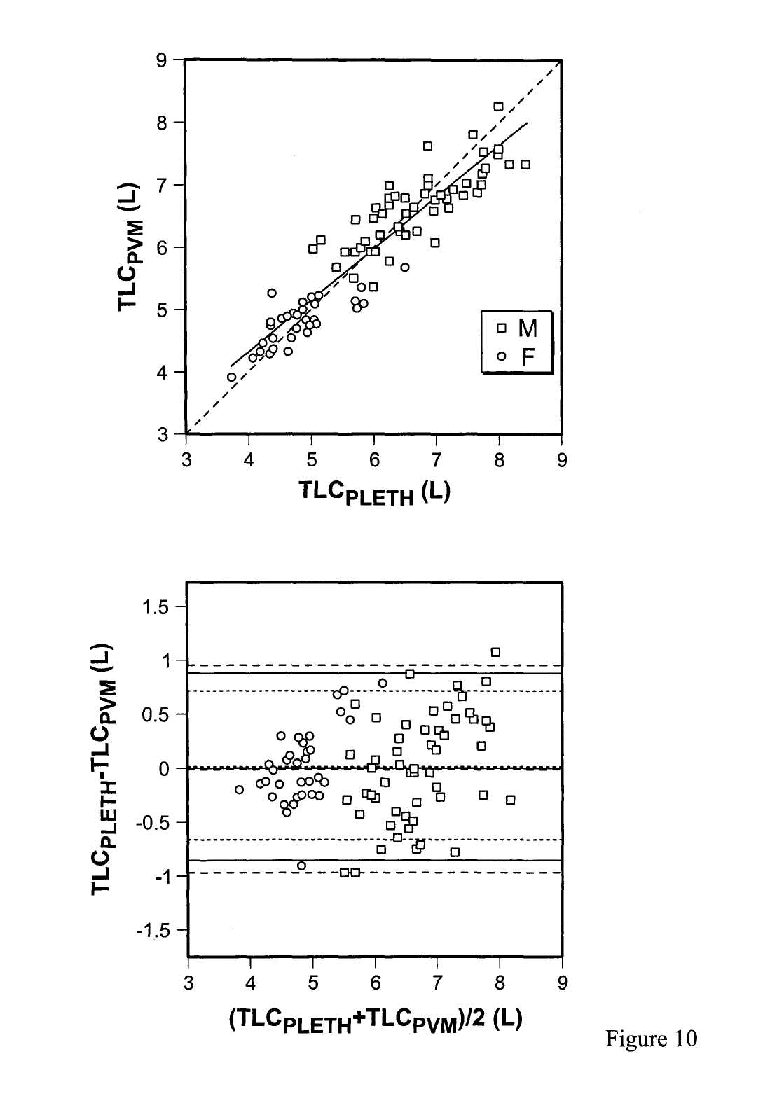

FIG. 10 illustrates a comparison of results of measured TLC in patients using the whole body plethysmography technique vs. results of measured TLC in the same patients using a controlled occlusion of airways technique with a breathing device, such as the breathing device 100, the breathing device 800, or other breathing device according to the present disclosure. For example, FIG. 10 illustrates scatter and Bland-Altman plots of TLC_{PLETH} (i.e., results of measured TLC in patients using the whole body plethysmography technique) and TLC_{PVM} (i.e., results of measured TLC in the same patients using a controlled occlusion of airways technique with a breathing device, such as the breathing device 100, the breathing device 800, or other breathing device according to the present disclosure) of a reference group of forty-seven (47) healthy subjects. As, shown, TLC_{PVM}=0.837·TLC_{PLETH}+0.97 L, r²=0.825 (p-value<0.01); R=0.925 (0.888, 0.95) (Pearson), residual standard deviation=0.397 L, standard deviation of TLC_{PLETH}−TLC_{PVM}=0.44 L, Coefficient of Variation (CV)=7.4%. The identity (dashed) and regression (solid) lines are shown for reference. Males and females are shown as squares and circles, respectively. Mean and limits of agreement lines (1.96STD) of the total, male (squares) and female (circles) populations are shown in solid, dashed and dotted lines, respectively at the bottom panel. Mean differences of the total, male and female populations are −0.001 L, −0.001 L, and 0.012 L, respectively.

A number of embodiments have been described. Nevertheless, it will be understood that various modifications may be made. For example, operations using the breathing device 100 and/or the breathing device 800 (or any other breathing device described herein) may include more steps or fewer steps than those described. Further, the steps described in such operations may be performed in different successions. Accordingly, other embodiments are within the scope of the following claims.

1. An apparatus, comprising:
a mouthpiece adapted to communicate an airflow between a user and an airflow chamber in fluid communication with the mouthpiece;
an interrupter in fluid communication with the airflow chamber and adapted to initiate an occlusion event by adjusting between an open state of the interrupter and a closed state of the interrupter, such that fluid communication is at least partially prevented between the airflow chamber and ambient air during the occlusion event initiated by the interrupter;
a container in fluid communication with the airflow chamber;
a flow rate sensor in fluid communication with the airflow chamber and adapted to measure an airflow exchange between the airflow chamber and the container during the occlusion event; and
a controller adapted to determine an instantaneous lung volume based on the measured airflow exchange between the airflow chamber and the container.

2. The apparatus of claim 1, wherein the controller is adapted to determine an instantaneous lung volume based only on the measured airflow exchange between the airflow chamber, container and ambient air.

3. The apparatus of claim 1, further comprising a pump in fluid communication with the container, wherein the pump is adapted to initiate an airflow between the container and the atmosphere.

4. The apparatus of claim 1, wherein the interrupter comprises a first interrupter, the apparatus further comprising a second interrupter disposed between the airflow chamber and the container.

5. The apparatus of claim 4, wherein the second interrupter is adapted to initiate an occlusion event by adjusting between an open state of the second interrupter and a closed state of the second interrupter, such that fluid communication is at least partially prevented between the airflow chamber and the container during the occlusion event initiated by the second interrupter.

6. The apparatus of claim 1, further comprising a first pressure sensor adapted to measure a static pressure of the container.

7. The apparatus of claim 6, wherein the pump is adapted to initiate an airflow from the container to the atmosphere when the first interrupter is in the open state and the second interrupter is in the closed state, such that the container is at a negative pressure relative to atmospheric.

8. The apparatus of claim 7, wherein the flow rate sensor is adapted to measure a first airflow exchange between the mouthpiece and the airflow chamber when the first interrupter is in the open state and the second interrupter is in the closed state, and
wherein the flow rate sensor is adapted to measure a second airflow exchange between the mouthpiece and the container when the second interrupter is adjusted from the closed state to the open state.

9. The apparatus of claim 8, wherein the controller is adapted to determine a difference between the first and second airflow exchanges.

10. The apparatus of claim 9, wherein the controller is adapted to determine a level of flow limitation of the user based on the determined difference between the first and second airflow exchanges at the negative pressure in the container.

11. The apparatus of claim 4, wherein at least one of the first or second interrupters comprises a shutter.

12. The apparatus of claim 6, further comprising:
a second pressure sensor disposed within the airflow chamber and adapted to measure a static pressure of the airflow chamber.

13. The apparatus of claim 12, wherein the controller is adapted to receive a plurality of pressure measurements from the first and second pressure sensors, and
wherein the controller is adapted to determine a density of the measured airflow exchange based on the plurality of pressure measurements.

14. The apparatus of claim 13, wherein the controller is adapted to adjust the determined instantaneous lung volume based on the determined density of the measured airflow exchange.

15. The apparatus of claim 4, wherein the first interrupter is adapted to adjust to a state near but not at the closed state to initiate the occlusion event.

16. The apparatus of claim 1, wherein the controller is further operable to determine a residual volume of air in the lungs of the user based on the determined instantaneous lung volume.

17. The apparatus of claim 16, wherein the controller is further operable to determine a total lung capacity of the user based on the determined residual volume of air in the lungs of the user.

18. The apparatus of claim 17, wherein the controller is further operable to determine a thoracic gas volume of the user based on the determined residual volume of air in the lungs of the user.

19. The apparatus of claim 1, wherein at least one of the airflow chamber or the container is adapted to be kept at substantially isothermal conditions.

20. A method, comprising:
receiving airflow through a conduit;
occluding the conduit for a predetermined time interval to substantially prevent airflow through the conduit;
measuring an airflow exchange between the conduit and a chamber in fluid communication with the conduit; and
determining an instantaneous lung volume based on the measured airflow exchange.

21. The method of claim 20, wherein determining an instantaneous lung volume based on the measured airflow exchange comprises determining an instantaneous lung volume based only on the measured airflow exchange.

22. The method of claim 20, further comprising:
initiating a first occlusion event for a first predetermined time interval to substantially prevent airflow through the conduit;
initiating a second occlusion event for a second predetermined time interval;
concluding the first occlusion event;
taking a plurality of pressure measurements in the conduit during the first and second occlusion events; and
determining an instantaneous lung volume based on the measured pressures.

23. The method of claim 20, further comprising initiating a third occlusion event between the conduit and the chamber such that fluid communication is at least partially prevented between the conduit and the chamber.

24. The method of claim 23, further comprising initiating an airflow between the chamber and the atmosphere during the third occlusion event.

25. The method of claim 24, further comprising:
taking a pressure measurement of the chamber during the third occlusion event;
concluding the third occlusion event; and
measuring an airflow rate through the chamber subsequent to concluding the third occlusion event.

26. The method of claim 25, further comprising:
determining a difference between the measured airflow exchange between the conduit and a chamber and the measured airflow rate through the chamber subsequent to concluding the third occlusion event.

27. The method of claim 26, further comprising:
determining a level of flow limitation of the user based on the determined difference at the pressure measurement of the chamber taken during the third occlusion event.

28. The method of claim 22, further comprising:
determining a density of the measured airflow exchange based on the plurality of pressure measurements.

29. The method of claim 28, further comprising:
adjusting the determined instantaneous lung volume based on the determined density of the measured airflow exchange.

30. The method of claim 20, further comprising determining a residual volume of air in the lungs of the user based on the determined instantaneous lung volume.

31. The method of claim 20, further comprising determining a total lung capacity of the user based on the determined residual volume of air in the lungs of the user.

32. The method of claim 20, further comprising determining a thoracic gas volume of the user based on the determined residual volume of air in the lungs of the user.

33. The method of claim 20, further comprising maintaining at least one of the conduit or the chamber at substantially isothermal conditions.
